(19) United States
(12) Patent Application Publication
(10) Pub. No.: US 2011/0201942 A1
(43) Pub. Date: Aug. 18, 2011

(54) DEVICE AND METHOD FOR OPTICALLY EXAMINING THE INTERIOR OF A BODY PART

(75) Inventors: Marius Iosif Boamfa, Eindhoven (NL); Wouter Harry Jacinth Rensen, Eindhoven (NL)

(73) Assignee: KONINKLIJKE PHILIPS ELECTRONICS N.V., EINDHOVEN (NL)

(21) Appl. No.: 13/125,677

(22) PCT Filed: Oct. 21, 2009
(86) PCT No.: PCT/IB2009/054652

§ 371 Date: Apr. 22, 2011

(30) Foreign Application Priority Data
Oct. 27, 2008 (EP) 08167620.7

(51) Int. Cl. A61B 6/00

(52) U.S. Cl. 600/476

(57) ABSTRACT

A device for optically examining the interior of a body part by transillumination is provided. The device comprises: an illumination unit (2) adapted for emitting polarized light (8) towards a body part (5) to be examined; and a detector unit (6) adapted for detecting light in transmission. A polarizer (10) is arranged in front of the detector unit (6).

### FIELD OF INVENTION

The present invention relates to a device for optically examining the interior of a body part and to a method for optically examining the interior of a body part.

### BACKGROUND OF THE INVENTION

In the context of the present application, the term light is to be understood to mean non-ionizing electromagnetic radiation, in particular with wavelengths in the range between 400 nm and 1400 nm. The term optically examining means examining by means of light. The term body part means a part of a human or animal body.

In recent years, several different types of devices for optically examining the interior of turbid media have been developed in which the turbid medium under examination, such as a body part, is illuminated with light from a light source and light emanating from the turbid medium is detected by a detector unit in transmission or reflection geometry. In such devices, the detected light is used to gather information about the interior of the turbid medium. Depending on the type of device for optically examining the interior of a turbid medium, e.g. two-dimensional or three-dimensional images of the interior of the turbid medium can be reconstructed or information about concentrations of different substances inside the turbid medium can be extracted from the detected light.

U.S. Pat. No. 5,415,655 shows a medical device for examining tissue by means of light. The medical device has a flexible light guide having a light energy input end adapted for connecting to a light energy source and a light energy output end. The light energy output end outputs a beam of light energy.

Recently, it has been suggested to use devices for optically examining the interior of turbid media to optically detect disease activity of joint diseases such as rheumatoid arthritis (RA) by illuminating both the joints and intermediate tissue of a body part under examination with light and detecting light emanating from the body part.

The treatment of such joint diseases is staged. Usually, a patient first receives pain killers. These are frequently followed by non-steroid anti-inflammatory drugs (NSAIDs) and disease modifying anti-rheumatic drugs (DMARDs). In many cases, the last stage in treatment with drugs is the use of biological therapies. In particular the last category is expensive and treatment can cost tens of thousands of dollars per year per patient. Additionally, the drugs used in later stages of treatment often cause more severe side effects. With respect to such joint diseases, medical professionals base their decisions on changes in therapy on disease activity which is given by the number and the severity of inflamed joints.

Since rheumatoid arthritis is a progressive disease and early diagnosis and start of treatment can help postponing adverse effects and high costs of treatment, there is a demand for methods and devices for providing satisfactory information about the condition of joints and which assist a medical professional to come to a conclusion with respect to the actual joint condition. Conventionally, rheumatologists use the so-called Disease Activity Score (DAS-28) for diagnosis and treatment monitoring. Since this method is time-consuming, operator-dependent, and has limited sensitivity, there is a demand for suitable devices for detecting disease activity. Use of devices for examining the respective body parts by means of light shows promising results as disease activity monitors.

According to a device for optically examining the interior of a body part by transillumination known to the applicant which device is specifically adapted for detecting disease activity of joint diseases, a body part, such as a human hand, is placed on a plate made of a transparent material. For examination, the body part is illuminated with an extended light source positioned below the plate and, in transmission geometry, light is detected by a detector unit situated on the opposite side of the turbid medium with respect to the light source. For example, the detector unit may be formed by a CCD camera. However, in such an arrangement, e.g. in a case in which the body part is a hand which is a typical situation for joint disease activity monitoring, light used for illuminating the body part will also be transmitted from the light source to the detector unit without passing through the body part. For example, the light will be transmitted between the fingers in the case of the body part being formed by a hand. Since such light will not have been attenuated in the body part, the intensity on the detector unit of this part of the light will be high as compared to the other part of the light which has passed through the body part. Thus, the light not having passed through the body part can saturate the detector unit such that, as a result, the relevant light which has passed through the body part can only be detected with less accuracy.

### SUMMARY OF THE INVENTION

It is an object of the present invention to provide a device for examining the interior of a body part by transillumination in which light attenuated by the body part can be detected with higher accuracy and unattenuated light is prevented from saturating the detector unit.

This object is solved by a device for optically examining the interior of a body part by transillumination according to claim 1. The device comprises: an illumination unit adapted for emitting polarized light towards a body part to be examined; and a detector unit adapted for detecting light in transmission. A polarizer is arranged in front of the detector unit. Thus, the device is adapted such that a body part under examination is illuminated with polarized light. Preferably, the polarized light is linearly polarized. Both light which has passed through the body part and light which has not passed through the part can arrive at the polarizer which is arranged in a transmission geometry. The part of the light arriving at the polarizer without having passed through the body part, i.e. a part of the light which still has the original polarization, can easily be blocked by the polarizer. In contrast, the polarization of the light which has passed through the body part will be different from the original polarization such that the appropriately set polarizer will not block this part of the light. As a consequence, only the light which has passed through the body part is allowed to pass on to the detector unit. Thus, the light which has passed through the body part can be detected with higher accuracy and unattenuated light is prevented from saturating the detector unit. Preferably, the device is specifically adapted for examining a body part comprising at least one joint.

Preferably, the illumination unit is adapted such that the polarized light is polarized in a first direction and the polarizer is arranged such that light polarized in the first direction is blocked. In this case, all the unattenuated light reaching the polarizer is reliably blocked and only the light which has passed through the body part can pass to the detector unit.

According to an aspect, the device comprises a support adapted for accommodating a body part to be examined. In this case, the position of the body part with respect to the illumination unit and to the polarizer is predetermined and thus accurate measurements are allowed for. Preferably, the support is adapted for accommodating a human hand as a body part to be examined. In this case, the device is particularly suited for diagnosis and treatment monitoring with respect to joint diseases such as rheumatoid arthritis.

According to one aspect, the illumination unit comprises the support and the support comprises a further polarizer for generating the polarized light. In this case, (less-expensive) light sources which do not provide polarized light can be used in the illumination unit and the polarized light for illumination is nevertheless achieved with a very compact arrangement.

If the illumination unit is adapted for emitting collimated light, adverse influences on the detected light due to depolarizing reflection on the border of the body part under examination are minimized.

If the illumination unit comprises at least one laser as a light source, no further polarizer is necessary for generating the polarized light since the laser emission is linearly polarized. The light source may for instance be formed by a single laser or by a laser array comprising a plurality of lasers.

Preferably, the device is a medical optical examination apparatus. According to one aspect, the device is adapted for optical detection of joint diseases. In this case, disease activity of e.g. rheumatoid arthritis can be conveniently monitored.

The object is also solved by a method for optically examining the interior of a body part by transillumination according to claim 10. The method comprises the steps: illuminating the body part with polarized light; directing light which has interacted with the body part and light which has not interacted with the body part to a polarizer; and detecting light which has passed through the polarizer. According to the method, the body part is illuminated with polarized light and both light which has passed through the body part and light which has not passed through the body part are directed to a polarizer. Only the light which is passed through the polarizer is detected. Thus, the body part to be examined can be illuminated over a wide area (or even as a whole) and, at the same time, due to the fact that only the light passing through the polarizer is detected, light attenuated by the body part can be detected with high accuracy and unattenuated light is prevented from saturating the detector unit used for detecting the light. Preferably, the body part under examination comprises at least one joint.

Preferably, the polarized light used for illuminating is polarized in a first direction and the analyzer polarizer is arranged such that light polarized in the first direction is blocked. In this case, all the unattenuated light reaching the polarizer is reliably blocked and only the light which has passed through the body part can pass to the detector unit.

Preferably, collimated light is used for illuminating the body part. In this case, adverse influences on the detected light due to depolarizing reflection on the border of the body part under examination are minimized.

According one aspect, at least one laser is used to generate the polarized light. In this case, no further polarizer is necessary for generating the polarized light, since the laser emits linearly polarized light. For example, a single laser may be used or an array of lasers.

### BRIEF DESCRIPTION OF THE DRAWINGS

Further features and advantages of the present invention will arise from the detailed description of embodiments with reference to the enclosed drawings.

FIG. 1 schematically shows a general set-up in a known device for optically examining the interior of a body part by transillumination.

FIG. 2 schematically shows the position of joints in a human hand as an example for a body part to be examined.

FIG. 3 schematically shows an embodiment of a device for optically examining the interior of a body part by transillumination.

### DETAILED DESCRIPTION OF EMBODIMENTS

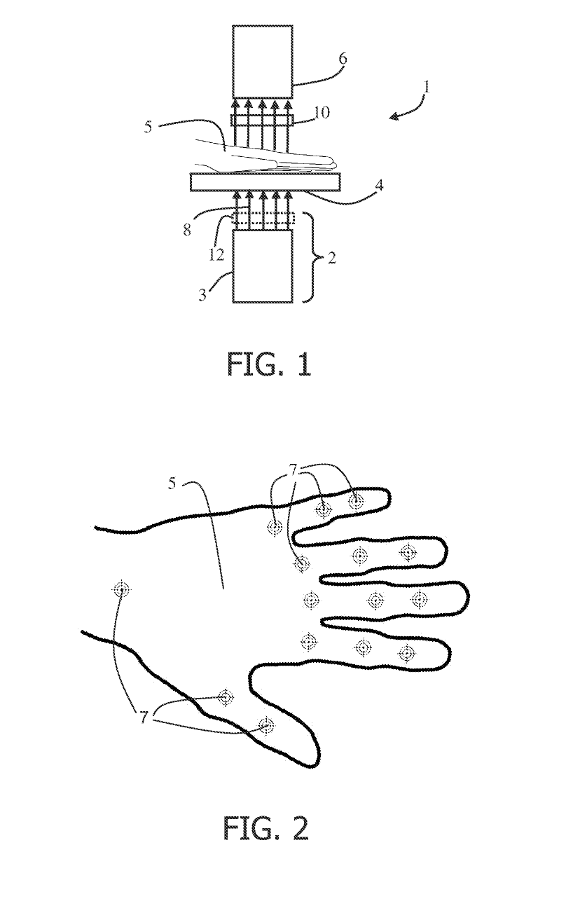

An embodiment of a device 1 for optically examining the interior of a body part by transillumination will be described with respect to FIG. 1.

The device is specifically adapted for examining the condition of joints. FIG. 1 schematically shows the set-up of such a device. As can be seen in FIG. 1, a body part 5 to be examined is placed on a transparent support 4. The support can e.g. be made from glass or a transparent plastic material. In the example, the body part 5 is formed by a human hand and the support 4 is formed by a transparent plate. FIG. 2 exemplary shows the regions of interest for joint disease activity monitoring, namely the joints 7 present in the body part 5. Although in the example shown in FIG. 2, the body part 5 is formed by a human hand, for examining the condition of joints, other body parts comprising at least one joint can be examined.

Below the support 4, i.e. on the opposite side of the support with respect to the body part 5, an illumination unit 2 emitting light 8 for illuminating the body part 5 is located. The illumination unit 2 comprises at least one light source 3 emitting the light used for illuminating. The light source 3 can e.g. be formed by a broadband light source, such as an incandescent lamp, or by a single-color light source such as an LED (light emitting diode) or a laser. A plurality of light sources 3 can be provided, e.g. an LED array or a laser array. According to the embodiment, the illumination unit 2 is adapted such that polarized light 8 is emitted. The emitted polarized light 8 has a first polarization direction. Preferably, linearly polarized light is emitted. For example, the polarized light 8 is generated by transmitting the light emitted by the light source 3 (or light sources) through an additional polarizer 12 (indicated by the dotted line in FIG. 1). In this case, the illumination unit 2 comprises at least one light source 3 and the additional polarizer 12. As an alternative, if a laser or a laser array is used for generating light, there is no need for a separate additional polarizer 12 since the laser light is already linearly polarized. Thus, in this case the illumination unit 2 does not comprise the additional polarizer 12.

Preferably, the illumination unit 2 is adapted to emit collimated light. This can e.g. be achieved by providing an additional collimator in the illumination unit 2 or by selection of an appropriate light source (or of appropriate light sources) emitting collimated light.

On the opposite side of the body part 5 as seen from the illumination unit 2, a detector unit 6 is arranged for detecting light emanating from the body part 5 (again schematically indicated by arrows). The detector unit 6 can e.g. be formed by a CCD camera or by another array of light detectors capable of acquiring a spatially resolved two-dimensional image. In front of the detector unit 6, as seen from the body part 5, a polarizer 10 is arranged. According to the embodiment, the polarizer 10 is arranged such that light having the first polarization direction, i.e. the polarization which light from the illumination unit 2 has, is blocked and light having a different polarization is allowed to pass. Thus, the polarizer 10 is arranged orthogonal to the polarization direction of the polarized light 8. For example, if the polarized light emitted by the illumination unit 2 is linearly polarized in a first direction, the polarizer 10 is arranged such that only light having a polarization in a direction orthogonal to the first direction is transmitted. For example, similar polarizers can be used for the polarizer 10 and the additional polarizer 12 and the polarizer 10 can be arranged to be rotated by 90 degrees about the optical axis with respect to the additional polarizer 12.

In the shown device, the whole body part 5 (and a part of the support 4) is illuminated by the illumination unit 2 with the polarized light 8 and the light arriving at the other side of the body part 5 is passed through the polarizer 10 before being detected by the detector unit 6 in a two-dimensionally resolved manner.

Since the polarized light 8 is used for illumination and the polarizer 10 is arranged in the light path in front of the detector unit 6, light which has not interacted with the body part 5 (and thus has kept the original polarization) will be blocked by the polarizer 10. Thus, this part of the light which would be likely to cause overexposure of the detector unit 6 is reliably blocked. In contrast, the part of the light which has traveled through the body part 5 has been subjected to multiple scattering and, as a result, lost the original polarization. As a consequence, the light which has traveled through the body part 5 is (partially) allowed to reach the detector unit 6 while the polarizer 10 blocks the rest of the light.

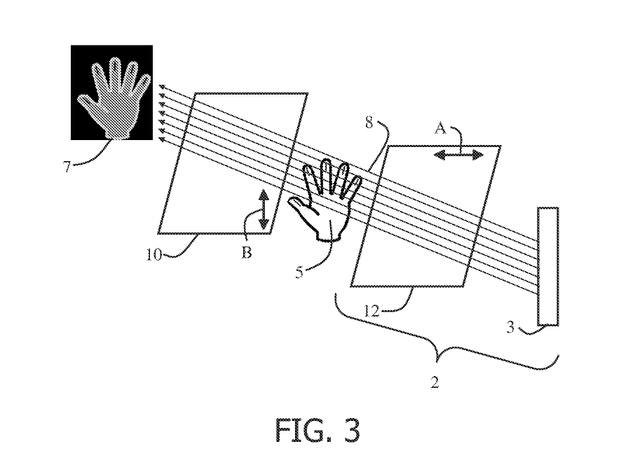

The working principle of the device 1 will be described again with respect to FIG. 3. It should be noted that some parts of the device are omitted in the schematic representation of FIG. 3. In particular, the support 4 is not shown in FIG. 3. Further, it should be noted that FIG. 3 shows the embodiment comprising the additional polarizer 12. However, as has been described above, in the case of one or more light sources 3 emitting polarized light 8 such as a laser or a laser array, the additional polarizer 12 can be dispensed with.

As can be seen in FIG. 3, light from the light source 3 is passed through the additional polarizer 12 which linearly polarizes the light in first direction A. The body part 5 to be examined is arranged such that the polarized light 8 impinges on the body part 5. The analyzer polarizer 10 is arranged behind the body part 5 in the light path. In other words, the body part 5 is arranged such that it is located between the illumination unit emitting polarized light and the polarizer 10 which is an analyzer polarizer. The polarizer 10 has a polarization direction B which is orthogonal to the first direction. The light which has passed through the body part 5 has lost the linear polarization due to the scattering processes and is therefore not blocked by the polarizer 10. As a consequence, the detector unit 6 only detects light which has passed through the body part 5.

As schematically indicated in FIG. 3, the border of the body part 5 under examination can appear slightly brighter in the detected image as compared to the rest of the body part 5. This effect is due to depolarizing reflection at the border. Such reflection causes a modification of the polarization state of the light resulting in the reflected light being partially allowed to pass the polarizer 10 and enter the detector unit 6. However, if collimated light is used for illuminating the body part 5, this effect can be significantly reduced since depolarizing reflection can be significantly suppressed. Thus, it is preferred that the illumination unit 2 is adapted for emitting collimated light.

To summarize, the embodiment provides a device and a method with which unattenuated light can reliably be prevented from reaching the detector unit 6 and light which has traveled through the body part 5 under examination can be detected with higher accuracy.

It should be noted that the position of the illumination unit 2 and the detector unit 6 can also be interchanged such that the support 4 is situated between the body part 5 and the detector unit 6. Further, it should be noted that the support 4 need not necessarily be provided as a separate unit but may also be integrated to the illumination unit 2 or to the detector unit 6. In the cases of light sources 3 requiring the additional polarizer 12, the additional polarizer 12 can be provided at different positions, e.g. between the light source 3 (or light sources) and the support 4, between the support 4 and the body part 5, or even integrated into the support 4. In the latter two cases, the support 4 will be considered to be a part of the illumination unit 2 emitting polarized light.

Preferably, the transparent support 4 is anti-reflection coated to avoid contrast-lowering reflections. In cases in which the support 4 is arranged such that polarized light impinges on the support (i.e. the additional polarizer 12 being arranged in the light path upstream of the support 4 or the light source 3 directly emitting polarized light), the support 4 is preferably adapted such that it comprises low birefringence.

The analyzer polarizer 10 need not be provided as a separate unit as shown in the Figures. For example, the polarizer 10 can also be provided immediately in front of the detector unit 6, for instance integrated or attached to the detector unit 6, or integrated or attached to an imaging lens system of the detector unit 6 (like in photo or video cameras). Further, in the case of the support 4 being arranged between the body part 5 and the detector unit 6, the polarizer 10 can also be integrated to the support 4.

1. Device for optically examining the interior of a body part by transillumination, the device comprising:
an illumination unit (2) adapted for emitting polarized light (8) towards a body part (5) to be examined; and
a detector unit (6) adapted for detecting light in transmission;
wherein a polarizer (10) is arranged in front of the detector unit (6).

2. Device according to claim 1, wherein the illumination unit (2) is adapted such that the polarized light (8) is polarized in a first direction (A) and the polarizer (10) is arranged such that light polarized in the first direction is blocked.

3. Device according to claim 1, wherein the device comprises a support (4) adapted for accommodating a body part (5) to be examined.

4. Device according to claim 3, wherein the support (4) is adapted for accommodating a human hand as a body part (5) to be examined.

5. Device according to claim 3, wherein the illumination unit (2) comprises the support and the support comprises an additional polarizer (12) for generating the polarized light.

6. Device according to claim 1, wherein the illumination unit (2) is adapted for emitting collimated light.

7. Device according to claim 1, wherein the illumination unit (2) comprises at least one laser as a light source (3).

8. Device according to claim 1, wherein the device is a medical optical examination apparatus.

9. Device according to claim 1, wherein the device is adapted for optical detection of joint diseases.

10. Method for optically examining the interior of a body part (5) by transillumination; the method comprising the steps:
illuminating the body part (5) with polarized light;
directing light which has interacted with the body part and light which has not interacted with the body part to a polarizer (10); and
detecting light which has passed through the polarizer (10).

11. Method according to claim 10, wherein the polarized light used for illuminating is polarized in a first direction (A) and the polarizer (10) is arranged such that light polarized in the first direction is blocked.

12. Method according to claim 10, wherein collimated light is used for illuminating the body part (5).

13. Method according to claim 10, wherein at least one laser is used to generate the polarized light.
